(19) United States
(12) Patent Application Publication
(10) Pub. No.: US 2026/0133108 A1
(43) Pub. Date: May 14, 2026

(54) METHOD FOR QUANTITATIVELY EVALUATING NEW-ASPHALT AND OLD-ASPHALT MIXING DEGREE BASED ON RECLAIMED ASPHALT MORTAR

(71) Applicant: South China University of Technology, Guangzhou (CN)

(72) Inventors: Yuan Zhang, Guangzhou (CN); Yefei Wang, Guangzhou (CN); Ke Pei, Guangzhou (CN); Jiangmiao Yu, Guangzhou (CN); Danqing Song, Guangzhou (CN); Nan Hu, Guangzhou (CN)

(21) Appl. No.: 19/445,451

(22) Filed: Jan. 9, 2026

(30) Foreign Application Priority Data
Aug. 10, 2025 (CN) 2025111128632

(51) Int. Cl. G01N 11/02; C08L 95/00; G01N 1/28; G01N 1/44; G01N 11/00; G01N 33/42

(52) U.S. Cl. CPC G01N 11/02; C08L 95/00; G01N 1/286; G01N 1/44; G01N 33/42; G01N 2001/2866; G01N 2011/0046; G01N 2011/006

(57) ABSTRACT

The present invention provides a method for quantitatively evaluating a blending degree of new- and old-asphalt based on recycled asphalt mortar. A linear viscoelastic area of the recycled asphalt mortar is tested and determined based on strain scanning; a rheological index of the recycled asphalt mortar is obtained based on frequency scanning; a difference between a rheological index of recycled asphalt mortar with complete blending of new asphalt and old asphalt and a rheological index of actually blended recycled asphalt mortar is compared; and a complex modulus deviation rate and the blending degree of new- and old-asphalt that are at a key frequency are calculated based on formulas

$$MDI = \frac{1}{N} {\sum }_{i - 1}^{N} \vert \frac{{G}_{actual blending ( {f}_{i} )}^{*} - {G}_{complete blending ( {f}_{i} )}^{*}}{{G}_{complete blending ( {f}_{i} )}^{*}} \vert$$

and DOB=1−MDI, where MDI represents the complex modulus deviation rate; DOB represents the blending degree of new- and old-asphalt; N represents a number of parallel tests

$${G}_{actual blending ( {f}_{i} )}^{*}$$

represents a complex modulus of the recycled asphalt mortar with actual blending of the new asphalt and the old asphalt; and

$${G}_{complete blending ( {f}_{i} )}^{*}$$

represents a complex modulus of the recycled asphalt mortar with complete blending of the new asphalt and the old asphalt. The present invention first evaluates the blending degree of new- and old-asphalt from the asphalt mortar, which breaks through the limitation of a traditional technical means in authenticity, accuracy practicability.

### TECHNICAL FIELD

The present invention relates to the technical field of road engineering, and in particular, to a method for quantitatively evaluating a blending degree of new- and old-asphalt based on recycled asphalt mortar.

### BACKGROUND

In a recycled asphalt mixture, a blending degree of new- and old-asphalt is one of the key factors that affect the performance recovery effect of a material. If new asphalt and old asphalt are not adequately blended, interfaces will be weak, and structures will be disassociated, thereby weakening the overall mechanical performance and long-term serviceability of the pavement. In current engineering practice, the evaluation of a recycling effect primarily relies on indirect methods such as macroscopic performance indicators of the pavement, including rutting test, low-temperature bending, water stability, or the like. Although these methods have particular reference values, they fail to reveal an actual blending state between the new asphalt and the old asphalt on the micro level.

On this background, it is particularly necessary to develop a method for evaluating a blending degree of new- and old-asphalt for recycled asphalt mortar. In one aspect, mortar serves as a key bonding structure that connects asphalt with fine aggregates, and can sensitively reflect an internal blending behavior of an asphalt phase. In another aspect, focusing on a mortar stage effectively shields the impact of an aggregate framework structure, so that asphalt phase blending characteristics can be observed more directly and clearly. By quantitatively analyzing diffusion, cross-linking, and interface transition characteristics of the new asphalt and the old asphalt in the mortar, a microscopic basis can be provided for quantitatively evaluating the activity of regenerants and optimizing heating and stirring processes, and the characteristics are used as leading indicators for predicting the performance of the overall pavement.

Current studies have attempted to characterize an asphalt phase blending process by using methods such as fluorescence microscopy, Fourier transform infrared spectroscopy (FTIR), and elemental analysis, but a standardized, engineering-oriented, and low-cost quantitative analysis system is still lacking.

Therefore, it is imperative to develop a method for evaluating a blending degree of new- and old-asphalt based on a recycled asphalt mortar system, so as to implement effective linkage from a microstructure to macro performance, thus providing a scientific support for the research, development, and application of recycled asphalt materials and promoting the development of sustainable road engineering.

### SUMMARY

In view of this, the present invention provides a method for quantitatively evaluating a blending degree of new- and old-asphalt based on recycled asphalt mortar, which breaks through the limitation of a traditional technical means in authenticity, accuracy, and practicability.

A technical solution in the present invention is implemented as follows:

In a first aspect, the present invention provides a method for quantitatively evaluating a blending degree of new- and old-asphalt based on recycled asphalt mortar, including the following steps:
step S1: determining use amounts of reclaimed asphalt pavement (RAP), a new aggregate, a new mineral filler, and new asphalt in a recycled asphalt mixture, and contents of an old aggregate, an old mineral filler, and old asphalt in the RAP;
preparing recycled asphalt mortar with complete blending of the new asphalt and the old asphalt and recycled asphalt mortar with actual blending of the new asphalt and the old asphalt based on the use amounts of the RAP, the new aggregate, the new mineral filler, and the new asphalt, and contents of the old aggregate, the old mineral filler, and the old asphalt in the RAP;
step S2: testing linear viscoelastic areas of the recycled asphalt mortar with complete blending of the new asphalt and the old asphalt and the recycled asphalt mortar with actual blending of the new asphalt and the old asphalt;
step S3: testing viscoelastic parameters of the recycled asphalt mortar with complete blending of the new asphalt and the old asphalt and the recycled asphalt mortar with actual blending of the new asphalt and the old asphalt based on the linear viscoelastic areas in step S2;
step S4: fitting complex modulus main curves of the recycled asphalt mortar with complete blending of the new asphalt and the old asphalt and the recycled asphalt mortar with actual blending of the new asphalt and the old asphalt based on the viscoelastic parameters in step S3, and calculating complex moduli; and
step S5: calculating a complex modulus deviation rate and the blending degree of new- and old-asphalt that are at a key frequency based on formulas

$$MDI = \frac{1}{N} {\sum }_{i - 1}^{N} \vert \frac{{G}_{actual blending ( {f}_{i} )}^{*} - {G}_{complete blending ( {f}_{i} )}^{*}}{{G}_{complete blending ( {f}_{i} )}^{*}} \vert and \\ DOB = 1 - MDI ,$$

where MDI represents the complex modulus deviation rate; DOB represents the blending degree of new- and old-asphalt; N represents a number of parallel tests;

$${G}_{actual blending ( {f}_{i} )}^{*}$$

represents a complex modulus of the recycled asphalt mortar with actual blending of the new asphalt and the old asphalt;

$${G}_{complete blending ( {f}_{i} )}^{*}$$

represents a complex modulus of the recycled asphalt mortar with complete blending of the new asphalt and the old asphalt;

Based on the above technical solution, further, the RAP in the recycled asphalt mortar with actual blending of the new asphalt and the old asphalt is added in a form of RAP mortar composed of the RAP old aggregate, the old asphalt, and the RAP old mineral filler; and the RAP mortar is obtained by finely sieving RAP with a particle size less than 2.36 mm.

Based on the above technical solution, further, in step S1, a method for preparing the recycled asphalt mortar with complete blending of the new asphalt and the old asphalt includes the following steps:
step A1: compounding the old asphalt from RAP in the recycled asphalt mixture and the new asphalt to obtain completely blended recycled asphalt;
step A2: heating the completely blended recycled asphalt in step A1 at 160° C. for 1 h, and heating the new aggregate, the RAP old aggregate, the RAP old mineral filler, and the new mineral filler at 170° C. for 3 h;
step A3: stirring the new aggregate and the RAP old aggregate in step A2 at 160° C. for 30 s, adding the completely blended recycled asphalt for agitation for 60 s, and adding the RAP old mineral filler and the new mineral filler for agitation for 120 s, wherein an agitation rate is 800 r/min;
Based on the above technical solution, further, in step A1, a method for preparing the old asphalt in the RAP includes performing recycling through an asphalt mixture analyzer and a rotary evaporator.

Based on the above technical solution, further, in step S1, a method for preparing the recycled asphalt mortar with actual blending of the new asphalt and the old asphalt includes the following steps:
step a1: heating the new aggregate at 170° C. for 3 h, heating the RAP mortar at 110° C. for 1 h, and heating the new asphalt at 160° C. until the new asphalt is in a flowing state; and
step a2: stirring the new aggregate and the RAP mortar in step a1 for 30 s, adding the new asphalt in step a1 for agitation for 60 s, and adding the new mineral filler for agitation for 120 s, wherein an agitation rate is 800 r/min.

Based on the above technical solution, further, step S2 includes: testing the linear viscoelastic areas of the recycled asphalt mortar with complete blending of the new asphalt and the old asphalt and the recycled asphalt mortar with actual blending of the new asphalt and the old asphalt based on strain scanning, and selecting a strain corresponding to a decrease of an initial value of a complex shear modulus G* to 95% as a critical point for determining the linear viscoelastic areas, to determine strain levels of the linear viscoelastic areas, wherein a strain scanning range has a logarithmic increase from 0.0001% to 0.1%; a scanning frequency is 10 Hz; and a temperature is 20° C.

Asphalt mortar is a typical viscoelastic material. When a strain or stress applied to a sample is within a range, a structure of the sample undergoes elastic deformation. This deformation can be fully recovered without causing damage to a material structure, thus exhibiting a linear viscoelastic response. A corresponding stress or strain range constitutes a linear viscoelastic area (LVE). However, when the applied stress or strain exceeds a threshold, the sample will undergo an irreversible structural change, thus exhibiting a nonlinear viscoelastic (NLVE) behavior. Typically, a boundary of the linear viscoelastic area is defined by a stress or strain corresponding to when a complex shear modulus (G*) decreases to 90% to 97% of its initial value. The present invention employs strain scanning to test the linear viscoelastic area of the recycled asphalt mortar, and selects the strain corresponding to a decrease of the initial value of G* to 95% as a key critical point for determining the linear viscoelastic area.

Based on the above technical solution, still further, step S3 includes: selecting corresponding strain amplitudes based on the linear viscoelastic areas, which are tested in step S2, of the recycled asphalt mortar with complete blending of the new asphalt and the old asphalt and the recycled asphalt mortar with actual blending of the new asphalt and the old asphalt, wherein a loading frequency has a logarithmic increase from 0.1 Hz to 10 Hz; and respectively testing, at 10° C., 20° C., 30° C., and 40° C., viscoelastic parameters of the recycled asphalt mortar with actual blending of the new asphalt and the old asphalt.

The purpose of setting a frequency range (0.1 Hz to 10 Hz) and a temperature range (10° C., 20° C., 30° C., and 40° C.) is to comprehensively capture viscoelastic behavior characteristics of the recycled asphalt mortar under various working conditions. This is to simulate a multi-scale loading environment under a realistic vehicle operation condition, systematically analyze the impact of the blending degree of new- and old-asphalt on the viscoelastic performance of the recycled asphalt mortar, and establish a performance identification model and blending degree index foundation within a broadband/wide temperature range.

Reasons for setting the frequency range include: (1) a loading frequency change: when traffic loading frequency change is simulated, if a frequency is small, a vehicle slowly passing through or long-time loading (such as a high-temperature slow lane and a traffic jam) is simulated; and if a frequency is large, rapid loading (such as a vehicle passing by speedily) is simulated. The frequency change can reflect the stress relaxation capability, modulus response, and rheological characteristic of the asphalt mortar under different stresses.

Reasons for setting the temperature range include: setting the four temperatures of 10° C., 20° C., 30° C., and 40° C., combined with the loading frequency range (0.1 Hz to 10 Hz), causes multi-temperature and multi-frequency data to cover a time-temperature space. This design lays a foundation for implementing translation superposition of multi-condition data and construction of the complex modulus main curve by using a time-temperature superposition principle (TTSP). It helps expand the frequency and temperature ranges and supports mechanical behavior modeling and performance prediction of a material under an actual service condition.

Based on the above technical solution, further, step S4 includes: calculating complex moduli of the recycled asphalt mortar with complete blending of the new asphalt and the old asphalt and the recycled asphalt mortar with actual blending of the new asphalt and the old asphalt at frequencies of 0.001 Hz, 1.0 Hz, and 1000 Hz.

Based on the above technical solution, further, step S5 includes: calculating complex modulus deviation rates and blending degrees of new- and old-asphalt of the recycled asphalt mortar with complete blending of the new asphalt and the old asphalt and the recycled asphalt mortar with actual blending of the new asphalt and the old asphalt at 0.001 Hz, 1.0 Hz, and 1000 Hz.

Based on the above technical solution, further, step S5 includes: calculating complex modulus deviation rates and blending degrees of new- and old-asphalt of the recycled asphalt mortar with complete blending of the new asphalt and the old asphalt and the recycled asphalt mortar with actual blending of the new asphalt and the old asphalt at 0.001 Hz.

Under a 0.001 Hz (high-temperature) condition, the complex modulus deviation rate (MDI) changes most significantly, which indicates that a performance difference caused by insufficient blending can be highlighted most. A medium frequency of 1 Hz is more suitable for being used as a “reference point” for performance evaluation. An MDI change at a high frequency of 1000 Hz mainly reflects low-temperature rigidity and a cracking risk.

Compared with the existing art, the present invention has the following beneficial effects:

The present invention provides a simpler and more reliable method for quantitatively evaluating a blending degree of new- and old-asphalt. The test method has high practicability, can quickly and accurately measure blending degrees of new- and old-asphalt of reclaimed asphalt mixture under different production conditions, to improve the production conditions, and has a high practical value.

By testing and determining a linear viscoelastic area of recycled asphalt mortar based on strain scanning, obtaining a rheological index of the recycled asphalt mortar based on frequency scanning, and comparing a difference between a rheological index of recycled asphalt mortar with complete blending of new asphalt and old asphalt and a rheological index of actually blended recycled asphalt mortar, the present invention provides complex modulus deviation rate and blending degree of new- and old-asphalt indexes to characterize the blending degree of new- and old-asphalt of the recycled asphalt mortar.

The present invention first evaluates the blending degree of new- and old-asphalt from the asphalt mortar, which breaks through the limitation of a traditional technical means in authenticity, accuracy practicability. By retaining an original state of RAP, considering a key factor of a mineral filler, and constructing a performance bridge between asphalt and pavement, the method provides a more reliable and more sensitivity scientific basis for design and quality control of the RAP and is particularly applicable to optimizing a recycling process and evaluating blending effectiveness under different RAP blending amounts.

### BRIEF DESCRIPTION OF DRAWINGS

To describe the technical solutions in the embodiments of the present invention or in the related art more clearly, the following briefly introduces the accompanying drawings for describing the embodiments or the related art. Apparently, the accompanying drawings in the following description show merely some embodiments of the present invention, and a person of ordinary skill in the art may still derive other drawings from the accompanying drawings without creative efforts.

FIG. 1 is a linear viscoelastic area of recycled asphalt mortar; and

FIG. 2 is a complex modulus main curve of different RAP blending amounts and blending degrees of new- and old-asphalt.

### DETAILED DESCRIPTION

The technical solutions in the embodiments of the present invention will be clearly and completely described below with reference to the embodiments of the present invention, and it is obvious that the described embodiments are only a part of the embodiments of the present invention but not all of them. All other embodiments obtained by a person of ordinary skill in the art based on the embodiments of present disclosure without making creative efforts shall fall within the protection scope of present disclosure.

In the present invention, RAP refers to a reclaimed asphalt pavement.

In the present invention, the stirring rate is 800 r/min.

In the present invention, “25% RAP” and “50% RAP” refer to addition of 25% or 50% RAP based on a mass fraction.

In the following specific implementations, among types of recycled asphalt mortar, matrix asphalt mortar is 0 RM; 25% RAP recycled asphalt mortar with actual blending of new asphalt and old asphalt is 25 RBM; 25% RAP recycled asphalt mortar with complete blending of new asphalt and old asphalt is 25 FBM; 50% RAP recycled asphalt mortar with actual blending of new asphalt and old asphalt is 50 RBM; and 50% RAP recycled asphalt mortar with complete blending of new asphalt and old asphalt is 50 FBM.

In the present invention, a content of asphalt in the recycled asphalt mortar is calculated based on RAP. The content of the asphalt in the recycled asphalt mortar is calculated by converting 0 RAP, 25% RAP, and 50% RAP used in the present invention. A commonly used optimal content of asphalt in an asphalt mixture is used for conversion, which is 4.4% by mass fraction. The use amount of the asphalt in the recycled asphalt mortar is 8.7% by mass fraction. When RAP blending amounts are 25% and 50%, a total use amount of the asphalt in the recycled asphalt mortar remains unchanged at 8.7%.

During preparation of recycled asphalt mortar with actual blending of new asphalt and old asphalt, the content of the asphalt in RAP mortar is 9.0% by mass. Based on a proportion relationship between old asphalt and new asphalt in the RAP mortar, a use amount of the new asphalt required to be added to the 25% RAP recycled asphalt mortar is 6.4% of the total asphalt mixture by mass fraction; and a use amount of the new asphalt required to be added to the 50% RAP recycled asphalt mortar is 4.2% by mass fraction.

The present invention provides a method for quantitatively evaluating a blending degree of new- and old-asphalt based on recycled asphalt mortar, including the following steps:
step S1: determining use amounts of RAP, a new aggregate, a new mineral filler, and new asphalt, and contents of an old aggregate, an old mineral filler, and old asphalt in the RAP;
preparing recycled asphalt mortar with complete blending of the new asphalt and the old asphalt and recycled asphalt mortar with actual blending of the new asphalt and the old asphalt based on the use amounts of the RAP, the new aggregate, the new mineral filler, and the new asphalt, and contents of the old aggregate, the old mineral filler, and the old asphalt in the RAP;
step S2: testing linear viscoelastic areas of the recycled asphalt mortar with complete blending of the new asphalt and the old asphalt and the recycled asphalt mortar with actual blending of the new asphalt and the old asphalt;
step S3: testing viscoelastic parameters of the recycled asphalt mortar with complete blending of the new asphalt and the old asphalt and the recycled asphalt mortar with actual blending of the new asphalt and the old asphalt based on the linear viscoelastic areas in step S2;
step S4: fitting complex modulus main curves of the recycled asphalt mortar with complete blending of the new asphalt and the old asphalt and the recycled asphalt mortar with actual blending of the new asphalt and the old asphalt based on the viscoelastic parameters in step S3, and calculating complex moduli; and
step S5: obtaining the blending degree of new- and old-asphalt based on formulas

$$MDI = \frac{1}{N} {\sum }_{i - 1}^{N} \vert \frac{{G}_{actual blending ( {f}_{i} )}^{*} - {G}_{complete blending ( {f}_{i} )}^{*}}{{G}_{complete blending ( {f}_{i} )}^{*}} \vert and \\ DOB = 1 - MDI ,$$

where MDI represents a complex modulus deviation rate; DOB represents the blending degree of new- and old-asphalt; N represents a number of parallel tests;

$${G}_{actual blending ( {f}_{i} )}^{*}$$

represents a complex modulus of the recycled asphalt mortar with actual blending of the new asphalt and the old asphalt;

$${G}_{complete blending ( {f}_{i} )}^{*}$$

represents a complex modulus of the recycled asphalt mortar with complete blending of the new asphalt and the old asphalt.

For example, at a frequency of 0.001 Hz (high temperature), an average complex modulus of 25RBM is 2.91E+07 Pa, and an average complex modulus of 25FBM is 2.33E+07 Pa. According to the formulas

$$MDI = \frac{1}{N} {\sum }_{i - 1}^{N} \vert \frac{{G}_{actual blending ( {f}_{i} )}^{*} - {G}_{complete blending ( {f}_{i} )}^{*}}{{G}_{complete blending ( {f}_{i} )}^{*}} \vert , DOB = 1 - MDI \\ MDI = \frac{( 2.91 E + 07 - 2.33 E + 07 )}{2.33 E + 07} = 24.8 \% and \\ DOB = 1 - 24.8 \% = 75.2 \%$$

are calculated and obtained.

In step S4, complex moduli at different RAP blending amounts and key frequencies are calculated.

An increase in an RAP blending amount means a decrease in an actual blending degree, while a high RAP blending amount means an increase in a proportion of the old asphalt added to the system. However, in actual construction, the old asphalt is often difficultly fully activated and mixed with the new asphalt. As a result, the blending is not complete; the content of effective asphalt in the system is reduced; and the structure is not uniform. It is manifested as an increase in the complex modulus G* and an increase in the complex modulus deviation rate (MDI) relative to a completely blended system.

The key frequencies are used to simulate responses under different service environments. Different frequencies simulate different working conditions, and are blending degree influencing features.

0.001 Hz (low frequency/high temperature): corresponding working conditions are rutting and slow loading. It is a most sensitive range for a viscoelastic behavior of a material. In this case, insufficient blending can significantly increase the viscosity of the system, causing a significantly high MDI index, which sensitively reflects a highlighting effect of macroscopic deformation potential and insufficient blending quality.

1 Hz (medium frequency/medium temperature): a corresponding working condition is a normal driving condition. It is a frequency range within which the material performance is relatively balanced. In this case, the MDI mainly reflects the consistency and uniformity of new- and old-asphalt blending and has strong indicative significance for the overall structural coordination of a material.

1000 Hz (high frequency/low temperature): a corresponding working conditions is an impact load or a low-temperature crack occurrence condition. A material response is predominantly rigid. In this frequency band, insufficient blending can lead to a significant increase in a complex modulus. It is manifested as abnormal structural rigidity, which can easily induce a brittle failure.

At 0.001 Hz or under a high-temperature condition, the complex modulus deviation rate (MDI) changes most significantly, which indicates that a performance difference caused by insufficient blending can be highlighted most. A medium frequency of 1 Hz is more suitable for being used as a “reference point” for performance evaluation. An MDI change at a high frequency of 1000 Hz mainly reflects low-temperature rigidity and a cracking risk.

### Embodiment 1

This embodiment provides a method for quantitatively evaluating a blending degree of new- and old-asphalt based on reclaimed asphalt, including the following steps:
Step S1: asphalt mortar with a 25% RAP blending amount was prepared, and 90 #matrix asphalt was used; and use amounts of an RAP old aggregate, old asphalt, an RAP old mineral filler, new asphalt, a new aggregate, and a new mineral filler were calculated based on the 25% RAP blending amount.

Recycled asphalt mortar with complete blending of the new asphalt and the old asphalt was composed of recycled asphalt with complete blending of the new asphalt and the old asphalt, the RAP old aggregate, the new aggregate, the RAP old mineral filler, and the new mineral filler. The use amount of the recycled asphalt used was 8.7% by mass fraction. The use amounts of the RAP old aggregate, the new aggregate, the RAP old mineral filler, and the new mineral filler were 21.9%, 64.9%, 0.9%, and 3.7% by mass fraction, respectively.

Recycled asphalt mortar with actual blending of the new asphalt and the old asphalt was composed of a new aggregate, RAP mortar, new asphalt, and a new mineral filler. Based on a proportion relationship between the old asphalt and the new asphalt in the RAP mortar, the use amount of the new asphalt that needs to be added into the 25% RAP recycled asphalt mortar was calculated to be 6.4% of the total recycled asphalt mortar by mass fraction. The use amounts of the new aggregate and the new mineral filler were 64.9%, and 3.7% by mass fraction, respectively.

A method for preparing the recycled asphalt mortar with complete blending of the new asphalt and the old asphalt includes the following steps:
Step A1: the old asphalt (which is recycled by an asphalt mixture analyzer and a rotary evaporator) in the reclaimed asphalt mixture material was compounded with the new asphalt in proportion, to prepare the homogeneous “completely blended recycled asphalt”.
Step A2: during preparation, the “completely blended recycled asphalt” was first heated at 160° C. for 1 h, and the new aggregate, the RAP old mineral filler, and the new mineral filler were heated at 170° C. for 3 h.
Step A3: then, at a blending temperature of 160° C., dry blending was performed on the preheated new aggregate and the RAP old aggregate for 30 s; the preheated “completely blended recycled asphalt” was then added for agitation for 60 s; and finally, the RAP old mineral filler and the new mineral filler were added for agitation for 120 s, to finally obtain the recycled asphalt mortar with complete blending of the new asphalt and the old asphalt.

A method for preparing the recycled asphalt mortar with actual blending of the new asphalt and the old asphalt includes the following steps:
Step a1: the new aggregate was heated at 170° C. for 3 h, the RAP mortar was heated at 110° C. for 1 h, and the new asphalt was heated at 160° C. until the new asphalt was in a flowing state.
Step a2: then, at a blending temperature of 160° C., dry blending was performed on the preheated new aggregate and the RAP mortar for 30 s; the flowing new asphalt was added for agitation for 60 s; and the new mineral filler was finally added for agitation for 120 s, to finally obtain the recycled asphalt mortar with actual blending of the new asphalt and the old asphalt.

A gradation design of the recycled asphalt mortar with the actual blending of the new asphalt and the old asphalt is shown in Table 1.

TABLE 1
Gradation design of the recycled asphalt mortar with actual
blending of the new asphalt and the old asphalt

| | | | | | | |
|---|---|---|---|---|---|---|
| Particle size of a | 2.36 | 1.18 | 0.6 | 0.3 | 0.15 | 0.075 |
| sieve pore | | | | | | |
| Percentage of | 100 | 66.9 | 47.1 | 33.3 | 23.2 | 8.5 |
| passing (%) | | | | | | |

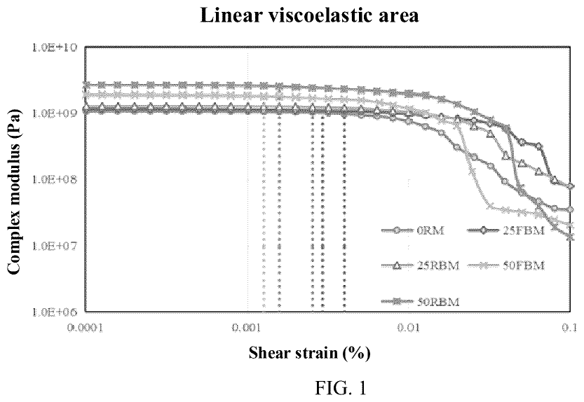

Step S2: strain scanning was used to test and determine a linear viscoelastic area (as shown in FIG. 1) of the recycled asphalt mortar, and a strain corresponding to a decrease of an initial value of a complex shear modulus G* to 95% as a key critical point for determining the linear viscoelastic area, to determine a strain level of the linear viscoelastic area. A strain scanning range had a logarithmic increase from 0.0001% to 0.1%; a scanning frequency was 10 Hz; and a temperature was set to 20° C.

Step S3: a strain amplitude of 0.001% was selected based on a linear viscoelastic area range of the recycled asphalt mortar that is determined by the strain scanning; a loading frequency was 0.1 Hz, which had a logarithmic increase to 10 Hz; and viscoelastic parameters of the asphalt mortar were respectively tested at 10° C., 20° C., 30° C., and 40° C.

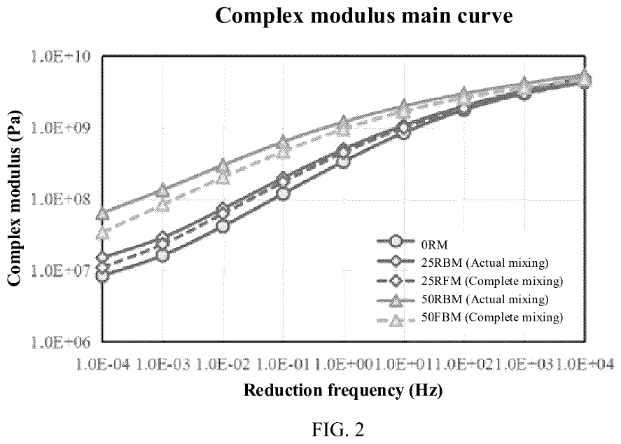

Step S4: Complex modulus main curves of the completely blended 25% RAP recycled asphalt mortar and the actually blended 25% RAP recycled asphalt mortar (as shown in FIG. 2) were fitted, and complex moduli of the completely blended 25% RAP recycled asphalt mortar and the actually blended 25% RAP recycled asphalt mortar at key frequencies of 0.001 Hz (high temperature), 1.0 Hz (medium temperature), and 1000 Hz (low temperature) were calculated.

Indexes of complex modulus deviation rate (MDI) and blending degree of new- and old-asphalt (DOB) at the key frequencies were calculated based on a formula, and the blending degree of new- and old-asphalt in the matrix asphalt mortar was quantitatively described.

### Embodiment 2

This embodiment provides a method for quantitatively evaluating a blending degree of new- and old-asphalt based on recycled asphalt. A difference between this embodiment and Embodiment 1 is that:
Step S1: asphalt mortar with a 50% RAP blending amount was prepared, and 90 #matrix asphalt was used; and use amounts of an RAP old aggregate, old asphalt, an RAP old mineral filler, new asphalt, a new aggregate, and a new mineral filler were calculated based on the 50% RAP blending amount.

Recycled asphalt mortar with complete blending of the new asphalt and the old asphalt was composed of recycled asphalt with complete blending of the new asphalt and the old asphalt, the RAP old aggregate, the new aggregate, the RAP old mineral filler, and the new mineral filler. The use amount of the recycled asphalt was 8.7% by mass fraction. The use amounts of the RAP old aggregate, the new aggregate, the RAP old mineral filler, and the new mineral filler were 37.4%, 45.8%, 8.2%, and 0% respectively.

Recycled asphalt mortar with actual blending of the new asphalt and the old asphalt was composed of a new aggregate, RAP mortar, new asphalt, and a new mineral filler. Based on a proportion relationship between the old asphalt and the new asphalt in the RAP mortar, the use amount of the new asphalt that needs to be added into the 50% RAP recycled asphalt mortar was calculated to be 4.2% of the total recycled asphalt mortar by mass fraction. The use amounts of the new aggregate and the new mineral filler were 45.8% and 0% respectively.

A gradation design of the recycled asphalt mortar with the actual blending of the new asphalt and the old asphalt is shown in Table 2.

TABLE 2
Gradation design of the recycled asphalt mortar with actual
blending of the new asphalt and the old asphalt

| | | | | | | |
|---|---|---|---|---|---|---|
| Particle size of a | 2.36 | 1.18 | 0.6 | 0.3 | 0.15 | 0.075 |
| sieve pore | | | | | | |
| Percentage of | 100 | 66.7 | 46.2 | 33.5 | 23.3 | 9.0 |
| passing (%) | | | | | | |

### Comparative Example 1

This comparative example provides a method for quantitatively evaluating a blending degree of new- and old-asphalt based on recycled asphalt, including the following steps:
matrix asphalt mortar was prepared, which was mainly composed of 70 #matrix asphalt, a new aggregate, and a new mineral filler. The 70 #matrix asphalt was used. By mass fraction, the use amount of the asphalt was 8.7%; the use amount of the new aggregate was 84%; and the use amount of the new mineral filler was 7.3%. A gradation design of the asphalt mortar is as shown in Table 3.

TABLE 3
Gradation design of the asphalt mortar

| | | | | | | |
|---|---|---|---|---|---|---|
| Particle size of a | 2.36 | 1.18 | 0.6 | 0.3 | 0.15 | 0.075 |
| sieve pore | | | | | | |
| Percentage of | 100 | 66.7 | 46.7 | 33.3 | 23.3 | 8.0 |
| passing (%) | | | | | | |

A method for preparing the matrix asphalt mortar includes the following steps:
Step a1: the new aggregate was heated at 170° C. for 3 h, and the matrix asphalt was heated at 160° C. until the matrix asphalt was in a flowing state.
Step a2: then, at a blending temperature of 160° C., the flowing matrix asphalt was first added for agitation for 60 s; and the new mineral filler was then added for agitation for 120 s, to finally obtain the matrix asphalt mortar.

Strain scanning was used to test and determine a linear viscoelastic area (as shown in FIG. 1) of the matrix asphalt mortar, and a strain corresponding to a decrease of an initial value of a complex shear modulus G* to 95% as a key critical point for determining the linear viscoelastic area, to determine a strain level of the linear viscoelastic area. A strain scanning range had a logarithmic increase from 0.0001% to 0.1%; a scanning frequency was 10 Hz; and a temperature was set to 20° C.

A rheological index of the matrix asphalt mortar was obtained based on frequency scanning. A strain amplitude of 0.001% was selected based on a linear viscoelastic area range of the recycled asphalt mortar that is determined by the strain scanning; a loading frequency was 0.1 Hz, which had a logarithmic increase to 10 Hz; and viscoelastic parameters of the matrix asphalt mortar were respectively tested at 10° C., 20° C., 30° C., and 40° C.

A complex modulus main curve of the matrix asphalt mortar (as shown in FIG. 2) was fitted, and complex moduli of the matrix asphalt mortar at key frequencies of 0.001 Hz (high temperature), 1.0 Hz (medium temperature), and 1000 Hz (low temperature) were calculated.

Indexes of complex modulus deviation rate (MDI) and blending degree of new- and old-asphalt (DOB) at the key frequencies were calculated based on a customized formula, and the blending degree of new- and old-asphalt in the matrix asphalt mortar was quantitatively described.

### Performance Detection

1. Extreme values of strains of the linear viscoelastic areas of the recycled asphalt mortar with complete blending of the new asphalt and the old asphalt, the recycled asphalt mortar with actual blending of the new asphalt and the old asphalt, and the matrix asphalt mortar in Embodiments 1 and 2 and the comparative example 1 are shown in FIG. 1 and Table 4.

TABLE 4
Extreme values of strains of the linear viscoelastic
areas of the recycled asphalt mortar

| Type of | Air | Initial complex | 95% of initial | Maximum |
|---|---|---|---|---|
| recycled | voids | modulus | complex modulus | shear strain |
| asphalt mortar | (%) | (Pa) | (Pa) | (Pa) |
| 0RM | 11.2 | 1.06E+09 | 1.01E+09 | 0.0029 |
| 25FBM | 11.1 | 1.15E+09 | 1.09E+09 | 0.0025 |
| 25RBM | 11.1 | 1.28E+09 | 1.21E+09 | 0.0036 |
| 50FBM | 11.3 | 1.88E+09 | 1.78E+09 | 0.0013 |
| 50RBM | 11.2 | 2.59E+09 | 2.46E+09 | 0.0016 |

From FIG. 1 and Table 4, it can be seen that the initial complex modulus of the recycled asphalt increases with the increase of the RAP mortar blending amount, and there is also a visible difference in modulus between the recycled asphalt mortar with complete blending of the new asphalt and the old asphalt and the recycled asphalt mortar with actual blending of the new asphalt and the old asphalt. The overall linear viscoelastic area decreases with the increase of the RAP mortar blending amount. To satisfy the linear viscoelastic areas of all tested mortar, a strain level of 0.001% should be selected for subsequent tests.

2. The complex moduli of the recycled asphalt mortar with actual blending of the new asphalt and the old asphalt, and the recycled asphalt mortar with complete blending of the new asphalt and the old asphalt in Embodiments 1 and 2 and the comparative example 1 at different RAP blending amounts and different frequencies are shown in Table 5. The complex modulus main curve is shown in FIG. 2.

TABLE 5
Complex moduli of the recycled asphalt
mortar at different frequencies

| | 25RBM | 25FBM | 50RBM | 50FBM |
|---|---|---|---|---|
| Frequency | (Pa) | (Pa) | (Pa) | (Pa) |
| 0.001 Hz (high | 2.91E+07 | 2.33E+07 | 1.35E+08 | 8.37E+07 |
| temperature) | | | | |
| 1.0 Hz (medium | 4.98E+08 | 4.47E+08 | 1.21E+09 | 9.55E+08 |
| temperature) | | | | |
| 1000 Hz (low | 3.29E+09 | 2.97E+09 | 4.17E+09 | 3.64E+09 |
| temperature) | | | | |

Table 5 shows average complex moduli of the recycled asphalt mortar at the key frequencies. At 0.001 Hz, 1.0 Hz, and 1000 Hz, respectively corresponding to a high temperature, a medium temperature, and a low temperature, the complex modulus of the recycled asphalt mortar with actual blending of the new asphalt and the old asphalt is compared with the complex modulus of the recycled asphalt mortar with complete blending of the new asphalt and the old asphalt.

3. The MDIs and DOBs of the recycled asphalt mortar with actual blending of the new asphalt and the old asphalt, and the recycled asphalt mortar with complete blending of the new asphalt and the old asphalt in Embodiments 1 and 2 and the comparative example 1 at different frequencies are shown in Table 6.

TABLE 6
MDIs and DOBs of the recycled asphalt
mortar at different frequencies

| | 25% RAP recycled | | 50% RAP recycled | |
|---|---|---|---|---|
| | asphalt mortar | | asphalt mortar | |
| Frequency | MDI | DOB | MDI | DOB |
| 0.001 Hz (high | 24.8% | 75.2% | 61.3% | 38.7% |
| temperature) | | | | |
| 1.0 Hz (medium | 11.3% | 88.7% | 26.6% | 73.4% |
| temperature) | | | | |
| 1000 Hz (low | 10.6% | 89.4% | 14.7% | 85.3% |
| temperature) | | | | |

According to Table 6, at 0.001 Hz (high temperature), the complex modulus deviation rates of the 25% RAP recycled asphalt mortar and the 50% RAP recycled asphalt mortar are maximum, which are 24.8% and 61.3% respectively, and corresponding blending degrees are 75.2% and 38.7% respectively.

At 1.0 Hz (medium temperature), an elastic component of the asphalt dominates a mechanical behavior. If blending of new- and old-asphalt is not sufficient, the brittleness of the old asphalt can form microcracks under cyclic loading and expand along a weak interface, thus accelerating a fatigue failure. The medium-temperature fatigue failure occurs at accumulation of multiple loads, while high-temperature ruts can be manifested under a short-term heavy load.

At 1000 Hz (low temperature), the complex modulus of the recycled asphalt mortar is mainly determined by an elastic modulus of the RAP old asphalt and a skeleton structure of the aggregate. The blending degree of new- and old-asphalt has a small impact on the low-temperature crack resistance. That is, although the complex modulus deviation rates of the 25% RAP recycled asphalt mortar with actual blending of the new asphalt and the old asphalt and the 50% RAP recycled asphalt mortar with actual blending of the new asphalt and the old asphalt are minimum at 1000 Hz, the blending degree of new- and old-asphalt is not a main impact factor.

In summary, it is more appropriate to define the blending degree of new- and old-asphalt at 0.001 Hz, i.e. under the high-temperature condition, as an effective blending degree.

The above describes the preferred embodiments of the present invention and is not intended to limit the present invention. Any modification, equivalent replacement, and improvement made within the spirit and scope of the present invention shall fall within the protection scope of the present invention.

What is claimed is:

1. A method for quantitively evaluating a blending degree of new- and old-asphalt based on recycled asphalt mortar, comprising the following steps:
step S1: determining use amounts of reclaimed asphalt pavement (RAP), a new aggregate, a new mineral filler, and new asphalt in the recycled asphalt mortar, and contents of an old aggregate, an old mineral filler, and old asphalt in the RAP;
preparing recycled asphalt mortar with complete blending of the new asphalt and the old asphalt and recycled asphalt mortar with actual blending of the new asphalt and the old asphalt based on the use amounts of the RAP, the new aggregate, the new mineral filler, and the new asphalt, and contents of the old aggregate, the old mineral filler, and the old asphalt in the RAP;
step S2: testing linear viscoelastic areas of the recycled asphalt mortar with complete blending of the new asphalt and the old asphalt and the recycled asphalt mortar with actual blending of the new asphalt and the old asphalt;
step S3: testing viscoelastic parameters of the recycled asphalt mortar with complete blending of the new asphalt and the old asphalt and the recycled asphalt mortar with actual blending of the new asphalt and the old asphalt based on the linear viscoelastic areas in step S2;
step S4: fitting complex modulus main curves of the recycled asphalt mortar with complete blending of the new asphalt and the old asphalt and the recycled asphalt mortar with actual blending of the new asphalt and the old asphalt based on the viscoelastic parameters in step S3, and calculating complex moduli; and
step S5: calculating a complex modulus deviation rate and the blending degree of new- and old-asphalt that are at a key frequency based on formulas
$$MDI = \frac{1}{N} {\sum }_{i - 1}^{N} \vert \frac{{G}_{actual blending ( {f}_{i} )}^{*} - {G}_{complete blending ( {f}_{i} )}^{*}}{{G}_{complete blending ( {f}_{i} )}^{*}} \vert and \\ DOB = 1 - MDI ,$$
wherein MDI represents the complex modulus deviation rate; DOB represents the blending degree of new- and old-asphalt; N represents a number of parallel tests;
$${G}_{actual blending ( {f}_{i} )}^{*}$$
represents a complex modulus of the recycled asphalt mortar with actual blending of the new asphalt and the old asphalt; and
$${G}_{complete blending ( {f}_{i} )}^{*}$$
represents a complex modulus of the recycled asphalt mortar with complete blending of the new asphalt and the old asphalt.
the RAP in the recycled asphalt mortar with actual blending of the new asphalt and the old asphalt is added in a form of RAP mortar composed of the RAP old aggregate, the old asphalt, and the RAP old mineral filler; and the RAP mortar is obtained by finely sieving an RAP material with a particle size less than 2.36 mm.
in step S1, a method for preparing the recycled asphalt mortar with complete blending of the new asphalt and the old asphalt comprises the following steps:
step A1: compounding the old asphalt and the new asphalt in the RAP in the recycled asphalt mortar to obtain completely blended recycled asphalt;
step A2: heating the completely blended recycled asphalt in step A1 at 160° C. for 1 h, and heating the new aggregate, the RAP old aggregate, the RAP old mineral filler, and the new mineral filler at 170° C. for 3 h; and
step A3: stirring the new aggregate and the RAP old aggregate in step A2 at 160° C. for 30 s, adding the completely blended recycled asphalt for agitation for 60 s, and adding the RAP old mineral filler and the new mineral filler for agitation for 120 s, wherein an agitation rate is 800 r/min;
in step S1, a method for preparing the recycled asphalt mortar with actual blending of the new asphalt and the old asphalt comprises the following steps:
step a1: heating the new aggregate at 170° C. for 3 h, heating the RAP mortar at 110° C. for 1 h, and heating the new asphalt at 160° C. until the new asphalt is in a flowing state; and
step a2: stirring the new aggregate and the RAP mortar in step a1 for 30 s, adding the new asphalt in step a1 for agitation for 60 s, and adding the new mineral filler for agitation for 120 s, wherein a stirring rate is 800 r/min.

2. The method for quantitatively evaluating the blending degree of new- and old-asphalt based on the recycled asphalt mortar according to claim 1, wherein step S2 comprises: testing the linear viscoelastic areas of the recycled asphalt mortar with complete blending of the new asphalt and the old asphalt and the recycled asphalt mortar with actual blending of the new asphalt and the old asphalt based on strain scanning, and selecting a strain corresponding to a decrease of an initial value of a complex shear modulus G* to 95% as a critical point for determining the linear viscoelastic areas, to determine strain levels of the linear viscoelastic areas, wherein a strain scanning range has a logarithmic increase from 0.0001% to 0.1%; a scanning frequency is 10 Hz; and a temperature is 20° C.

3. The method for quantitatively evaluating the blending degree of new- and old-asphalt based on the recycled asphalt mortar according to claim 1, wherein step S3 comprises: selecting corresponding strain amplitudes based on the linear viscoelastic areas, which are tested in step S2, of the recycled asphalt mortar with complete blending of the new asphalt and the old asphalt and the recycled asphalt mortar with actual blending of the new asphalt and the old asphalt, wherein a loading frequency has a logarithmic increase from 0.1 Hz to 10 Hz; and respectively testing, at 10° C., 20° C., 30° C., and 40° C., viscoelastic parameters of the recycled asphalt mortar with actual blending of the new asphalt and the old asphalt.

4. The method for quantitatively evaluating the blending degree of new- and old-asphalt based on the recycled asphalt mortar according to claim 1, wherein step S4 comprises: calculating complex moduli of the recycled asphalt mortar with complete blending of the new asphalt and the old asphalt and the recycled asphalt mortar with actual blending of the new asphalt and the old asphalt at frequencies of 0.001 Hz, 1.0 Hz, and 1000 Hz.

5. The method for quantitatively evaluating the blending degree of new- and old-asphalt based on the recycled asphalt mortar according to claim 1, wherein step S5 comprises: calculating complex modulus deviation rates and blending degrees of new- and old-asphalt of the recycled asphalt mortar with complete blending of the new asphalt and the old asphalt and the recycled asphalt mortar with actual blending of the new asphalt and the old asphalt at 0.001 Hz, 1.0 Hz, and 1000 Hz.

6. The method for quantitatively evaluating the blending degree of new- and old-asphalt based on the recycled asphalt mortar according to claim 5, wherein step S5 comprises: calculating complex modulus deviation rates and blending degrees of new- and old-asphalt of the recycled asphalt mortar with complete blending of the new asphalt and the old asphalt and the recycled asphalt mortar with actual blending of the new asphalt and the old asphalt at 0.001 Hz.
